(19) United States
(12) Patent Application Publication
(10) Pub. No.: US 2024/0000380 A1
(43) Pub. Date: Jan. 4, 2024

(54) WEARABLE DEVICE

(71) Applicant: PixArt Imaging Inc., Hsin-Chu City (TW)

(72) Inventors: Ming Shun Manson Fei, Hsin-Chu City (TW); CONG-JUN XIA, Hsin-Chu City (TW); GUI-PING SU, Hsin-Chu City (TW); HUA ZHANG, Hsin-Chu City (TW); Yen-Min Chang, Hsin-Chu City (TW); Chi-Yu Wu, Hsin-Chu City (TW)

(73) Assignee: PixArt Imaging Inc., Hsin-Chu City (TW)

(21) Appl. No.: 18/118,152

(22) Filed: Mar. 7, 2023

Related U.S. Application Data
(60) Provisional application No. 63/357,009, filed on Jun. 30, 2022.

(30) Foreign Application Priority Data
Dec. 27, 2022 (CN) 202211680498.1

(51) Int. Cl. A61B 5/00; A61B 5/0225

(52) U.S. Cl. CPC A61B 5/6802; A61B 5/02255

(57) ABSTRACT

A wearable device includes a case and a detection module. The case includes an inner casing and an outer casing. The inner casing includes a main body and a lateral wall. The main body is an annular structure. The lateral wall is disposed on a lateral side of the main body. The main body has a first installation area, a second installation area and a third installation area. The outer casing is disposed around the inner casing and abuts against the lateral wall, and the outer casing has a first opening. The detection module is disposed inside the case. The detection module includes an energy storage unit, an information transmission unit and an optical identification assembly. The energy storage unit is located on the first installation area. The information transmission unit is located on the second installation area. The optical identification assembly is located on the third installation area.

### CROSS REFERENCE TO RELATED APPLICATIONS

This application claims the benefit of U.S. Provisional Application No. 63/357,009, filed on Jun. 30, 2022. The content of the application is incorporated herein by reference.

### BACKGROUND OF THE INVENTION

### 1. Field of the Invention

The present invention relates to a wearable device, and more particularly, to a wearable device with an optical finger navigation function.

### 2. Description of the Prior Art

A conventional physiological information sensing device is mainly disposed on the mobile phone or the watch, but the user may not be able to wear the mobile phone or the watch in some conditions; for example, when the user sleeps, the item what the user wears should be as small as possible so that the user don't feel uncomfortable. In addition to the mobile phone and the watch, a conventional wearable electronic product can provide a smart ring, and the optical sensor and the light source inside the smart ring are applied for rotation detection and/or motion detection. However, the smart ring still needs some electronic components with auxiliary functions, such as the energy storage component, the charging component and the information transmission component. Therefore, design of a smart ring of accurately disposing the electronic components into a small space is an important issue in the optical wearable product industry.

### SUMMARY OF THE INVENTION

The present invention provides a wearable device with an optical finger navigation function for solving above drawbacks.

According to the claimed invention, a wearable device includes a case and a detection module. The case has an accommodating space where inside an organism is accommodated. The case includes an inner casing and an outer casing. The inner casing includes a main body and a lateral wall. The main body is an annular structure to form the accommodating space. The lateral wall is disposed on a lateral side of the main body. The main body has a first installation area, a second installation area and a third installation area. The outer casing is disposed around the inner casing and abuts against the lateral wall, and the outer casing has a first opening. The detection module is disposed inside the case. The detection module includes an energy storage unit, an information transmission unit and an optical identification assembly. The energy storage unit is located on the first installation area. The information transmission unit is located on the second installation area and adapted to transmit identification information. The optical identification assembly is located on the third installation area and electrically connected with the energy storage unit and the information transmission unit. The optical identification assembly aligns with the first opening and is adapted to output an optical detection signal toward the organism through the first opening and further to receive and transform an optical reflection signal from the organism into the identification information.

According to the claimed invention, the case further includes a lateral cover adapted to cover a space formed between the inner casing and the outer casing assembled with each other.

According to the claimed invention, the optical identification assembly includes an optical emitter, an optical receiver, a lens unit and a first sheltering unit. The lens unit is disposed above the optical emitter and the optical receiver, and used to constrain a transmission direction of the optical detection signal and the optical reflection signal, and the first opening is sealed by the first sheltering unit.

According to the claimed invention, the wearable device further includes a circuit board disposed on the inner casing and covered by the outer casing. The circuit board has several regions respectively corresponding to the first installation area, the second installation area and the third installation area. The detection module is disposed on a surface of the circuit board to face toward one of inside and outside of the case, or is disposed on two opposite surfaces of the circuit board to face toward the inside and the outside of the case.

According to the claimed invention, the circuit board is a plurality of hard circuit boards electrically connected with each other via transmission cables, or a plurality of soft circuit boards electrically connected with each other via the transmission cables, or the plurality of hard circuit boards and the plurality of soft circuit boards electrically connected with each other via the transmission cables. The wearable device further comprises a resiliently supporting component disposed under the soft circuit boards.

According to the claimed invention, the outer casing is made of metallic material, and the outer casing further includes a second opening corresponding to the information transmission unit. The outer casing further includes a second sheltering unit adapted to seal the second opening.

According to the claimed invention, a part of the outer casing that corresponds to the information transmission unit is made of non-metallic material. The detection module further includes a waterproof unit filled inside a gap between the optical identification assembly and the first opening.

According to the claimed invention, the detection module further includes a force feedback unit electrically connected with the optical identification assembly and adapted to move the optical identification assembly. The optical identification assembly is moved by material property of piezoelectric material, elastic material, or magnetic material of the force feedback unit.

According to the claimed invention, the main body of the inner casing includes a hole, and the wearable device further includes a charging assembly, and at least one pin of the charging assembly is inserted into the hole. A first magnetic component of the charging assembly is matched with a second magnetic component of an external charger to constrain a relative position between the wearable device and the external charger.

According to the claimed invention, the wearable device further includes a gravity sensor disposed one the main body and adapted to detect acceleration variation of the wearable device. The wearable device further includes a touch sensor disposed inside the inner casing and adapted to detect whether the organism is accommodated inside the case. The wearable device further includes a plurality of touch sensors arranged inside the inner casing along a horizontal direction of the case and adapted to detect a moving direction of the case relative to the organism.

According to the claimed invention, an included angle between a first virtual extension line from the optical identification assembly to a center of the case and a second virtual extension line from the information transmission unit to the center is ranged between sixty-five degrees and eighty degrees.

According to the claimed invention, a first engaging portion is disposed on a lateral side of the outer casing, and a second engaging component is disposed on the lateral wall of the inner casing, and the first engaging portion is engaged with an outer edge of the second engaging component to avoid separation. Waterproof material is filled between the first engaging portion and the second engaging component.

The wearable device of the present invention can be worn on the finger, the wrist or the forehead of the user for executing the optical identification function, and can optionally include the information transmission unit, the energy storage unit, the gravity sensor and the touch sensor to provide various detection functions. The optical identification assembly can detect rotation of the wearable device, and further be integrated with the photoplethysmography sensor and the pulse oximetry sensor for the biological detection.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram of a wearable device and an external charger according to an embodiment of the present invention.

FIG. 2 is a diagram of the wearable device in another view according to the embodiment of the present invention.

FIG. 3 is an exploded diagram of a case according to the embodiment of the present invention.

FIG. 4 and FIG. 5 are diagrams of a detection module in different views according to the embodiment of the present invention.

FIG. 6 is an exploded diagram of an optical identification assembly according to the embodiment of the present invention.

FIG. 7 is a section view of the wearable device according to the embodiment of the present invention.

FIG. 8 is a diagram of a part of the wearable device according to the embodiment of the present invention.

FIG. 9 is a section view of the wearable device according to the embodiment of the present invention.

FIG. 10 is another section view of the wearable device according to the embodiment of the present invention.

### DETAILED DESCRIPTION

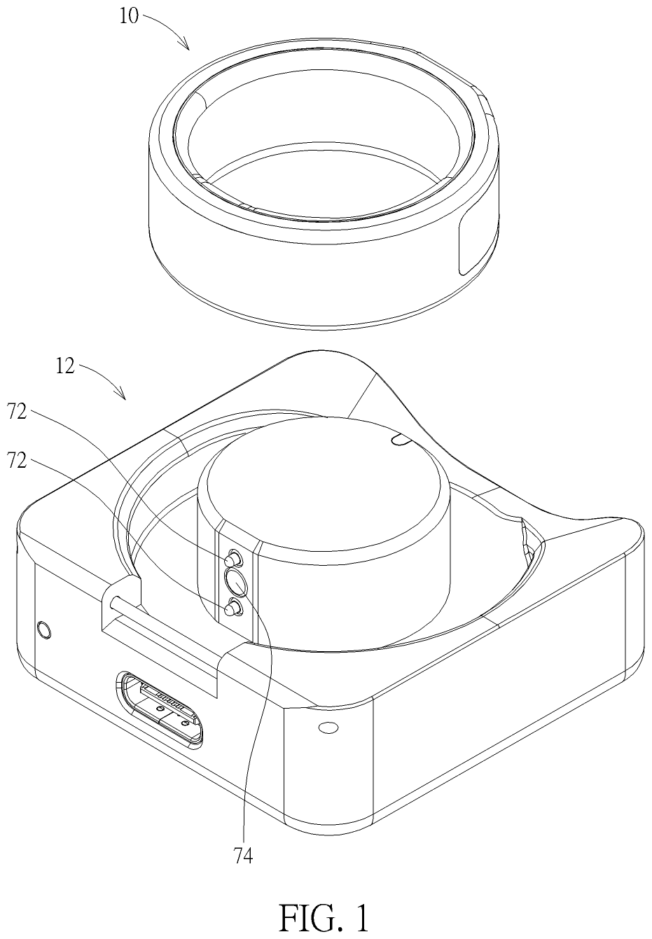

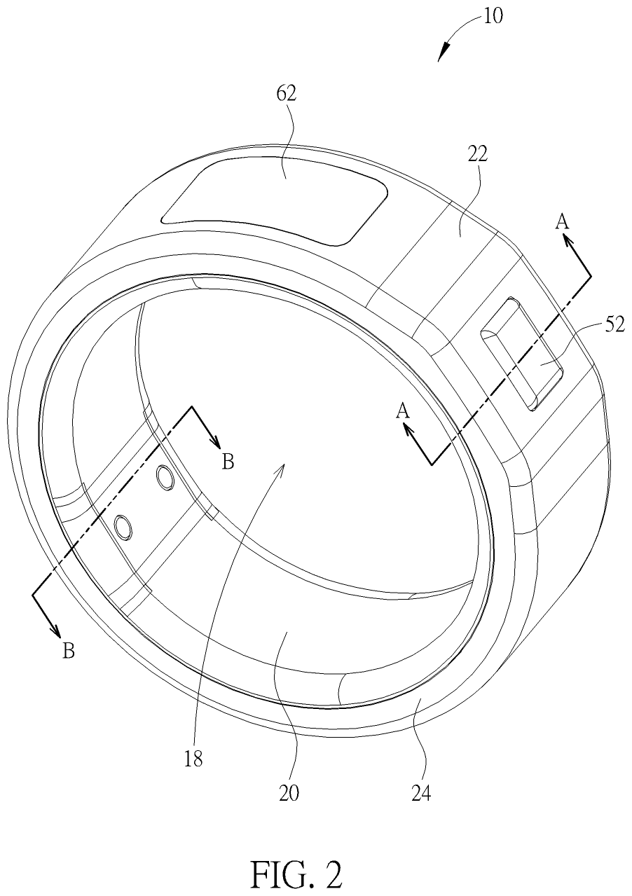

Please refer to FIG. 1 and FIG. 2. FIG. 1 is a diagram of a wearable device 10 and an external charger 12 according to an embodiment of the present invention. FIG. 2 is a diagram of the wearable device 10 in another view according to the embodiment of the present invention. The wearable device 10 can be a smart ring or other application, such as a smart wristband or a smart headband, and the application of the wearable device 10 depends on an actual demand. The wearable device 10 can detect whether the wearable device 10 is worn by the user, or detect whether the wearable device 10 is worn on the right hand or the left hand of the user, or detect whether the wearable device 10 is worn in a positive direction or in a negative direction. The wearable device 10 can further provide a finger navigation function, a finger pressing function, a heart rate detection function, a blood oxygen detection function and/or a motion detection function. The wearable device 10 can be charged by the by the external charger 12 via wireless charging technology, and have an artistic outward appearance.

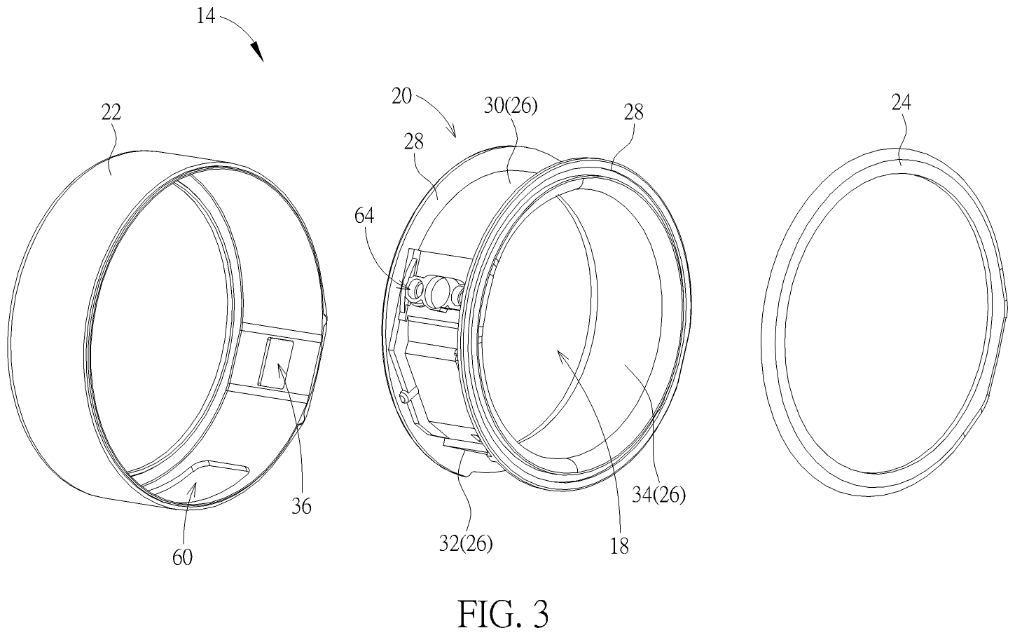

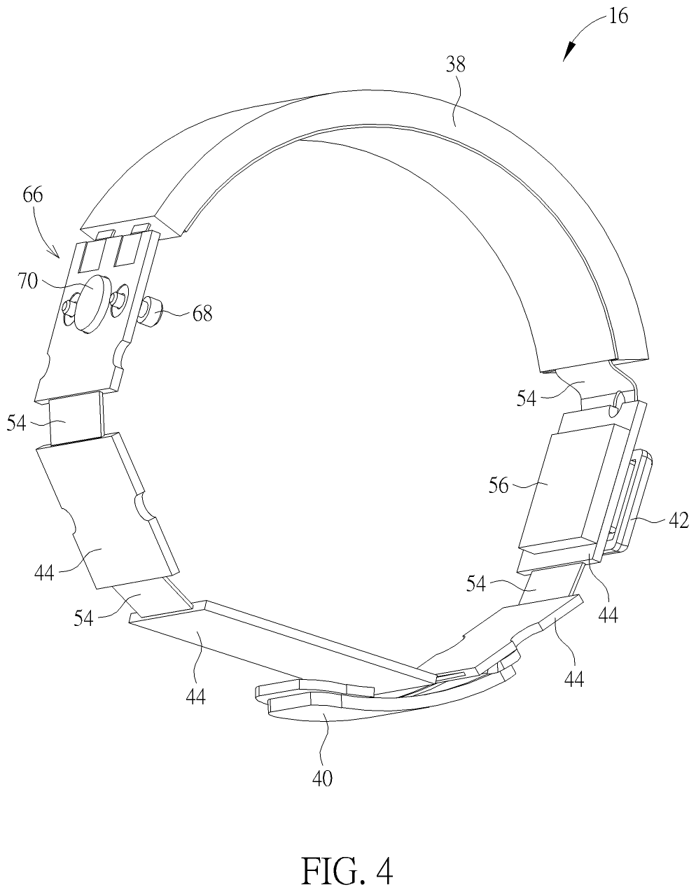

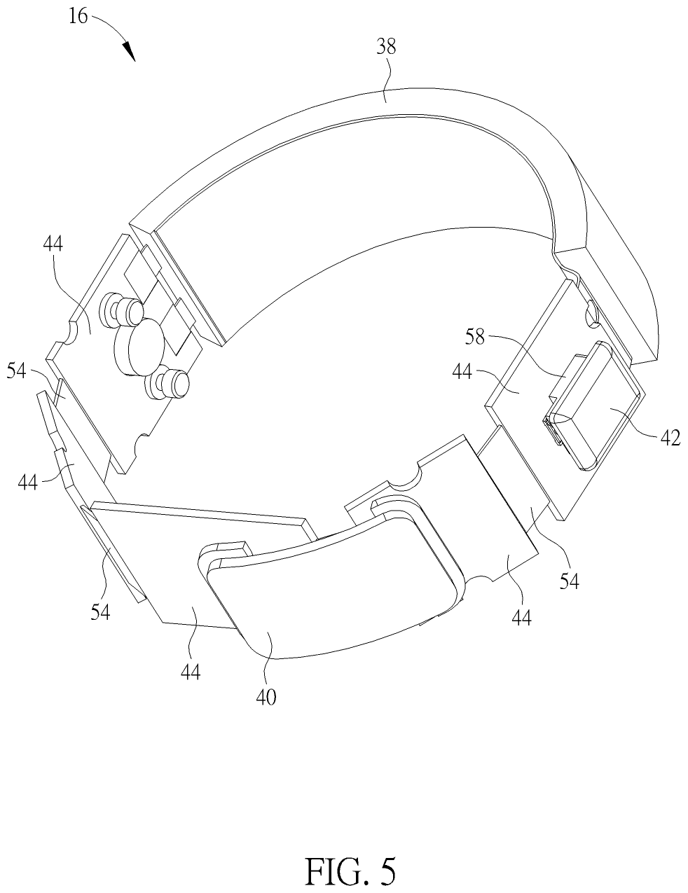

Please refer to FIG. 3 to FIG. 5. FIG. 3 is an exploded diagram of a case 14 according to the embodiment of the present invention. FIG. 4 and FIG. 5 are diagrams of a detection module 16 in different views according to the embodiment of the present invention. The wearable device 10 can include the case 14 and the detection module 16. The case 14 can have an accommodating space 18 where inside an organism is accommodated. The organism can be the user's finger if the wearable device 10 is the smart ring. The case 14 can include an inner casing 20, an outer casing 22 and a lateral cover 24. The detection module 16 can be disposed inside the inner casing 20. The outer casing 22 can be engaged with the inner casing 20 to cover and protect the detection module 16. A space formed between the inner casing 20 and the outer casing 22 can be covered by the lateral cover 24 to prevent the detection module 16 from being polluted by dust or liquid.

The inner casing 20 can include a main body 26 and a lateral wall 28. The main body 26 can be an annular structure used to form the accommodating space 18. The lateral wall 28 can be disposed on two lateral sides of the main body 26, and used as a retaining wall to be an interface engaged with the outer casing 22. The main body 26 can at least include a first installation area 30, a second installation area 32 and a third installation area 34. A side of the installation area can correspond to a size of each corresponding unit installed on the detection module 16. A number of the installation area can correspond to a number of the units installed on the detection module 16. The outer casing 22 can be disposed around the inner casing 20 and abut against or be engaged with the lateral wall 28. The outer casing 22 can at least include a first opening 36. The space can be formed between the inner casing 20 and the outer casing 22 when the inner casing 20 is engaged with the outer casing 22, and the lateral cover 24 can cover the space.

The detection module 16 can at least include an energy storage unit 38, an information transmission unit 40, an optical identification assembly 42 and a circuit board 44. The circuit board 44 can be disposed on the inner casing 20 and b covered by the outer casing 22. The circuit board 44 can have several regions respectively corresponding to the first installation area 30, the second installation area 32 and the third installation area 34 of the main body 26. The energy storage unit 38 can be disposed on a relevant region of the circuit board 44 and located on the first installation area 30. The information transmission unit 40 can be disposed on a relevant region of the circuit board 44 and located on the second installation area 32. The optical identification assembly 42 can be disposed on a relevant region of the circuit board 44 and located on the third installation area 34.

The energy storage unit 38 can store energy provided from the external charger 12, and transmit the energy to each electronic unit of the detection module 16. The information transmission unit 40 can transmit and receive information, such as identification information acquired by the optical identification assembly 42 or control information output by an external device. The optical identification assembly 42 can be electrically connected with the energy storage unit 38 and the information transmission unit 40. The optical identification assembly 42 can align with the first opening 36 on the outer casing 22, and used to emit the optical detection signal. The optical detection signal can be projected onto the organism through the first opening 36. The optical identification assembly 42 can further receive the optical reflection signal from the organism and transform the optical reflection signal into the identification information.

In the embodiment of the present invention, the electronic units of the detection module 16 can be disposed on one surface of the circuit board 44, such as an outer surface of the circuit board 44 facing the outer casing 22; in other possible embodiments, the electronic units of the detection module 16 may be respectively disposed two opposite surfaces of the circuit board 44, such as the outer surface of the circuit board 44 facing the outer casing 22, and an inner surface of the circuit board 44 facing the inner casing 20. Besides, according to annular design of the wearable device 10, the circuit board 44 can include a plurality of hard circuit boards electrically connected with each other via transmission cables 54, or a plurality of soft circuit boards electrically connected with each other via the transmission cables 54, or the plurality of hard circuit boards and the plurality of soft circuit boards electrically connected with each other via the transmission cables 54.

For example, the number of the electronic units included by the detection module 16 can depend on the actual design, and is not limited to the embodiment shown in FIG. 4. If the detection module 16 includes the plurality of optical identification assemblies 42, the plurality of optical identification assemblies 42 may be optionally disposed on the relevant regions of the circuit board 44 different from the first installation area 30 and the second installation area 32. In addition, the plurality of optical identification assemblies 42 can be further optionally disposed on position of the circuit board 44 facing at least one of the inside and the outside of the case 14, which means the plurality of optical identification assemblies 42 can be disposed on one surface or two opposite surfaces of the circuit board 14. When the optical identification assembly 42 is disposed on the circuit board 44 and faces toward the inside of the case 14, the optical identification assembly 42 can be used as one of a photoplethysmography (PPG) sensor and a pulse oximetry (SpO2) sensor, and provide a biological detection function. When the optical identification assembly 42 is disposed on the circuit board 44 and faces toward the outside of the case 14, the optical identification assembly 42 can provide the finger navigation function, the finger pressing function and the motion detection function.

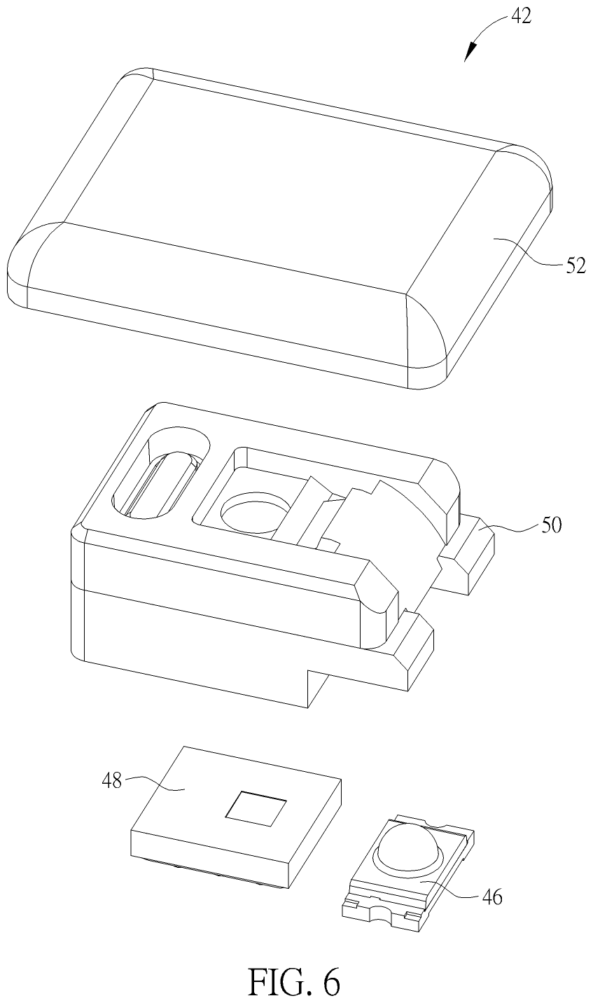

Please refer to FIG. 6. FIG. 6 is an exploded diagram of the optical identification assembly 42 according to the embodiment of the present invention. The optical identification assembly 42 can include an optical emitter 46, an optical receiver 48, a lens unit 50 and a first sheltering unit 52. The optical emitter 46 and the optical receiver 48 can be disposed adjacent to each other. The lens unit 50 can be disposed above the optical emitter 46 and the optical receiver 48, and used to constrain the transmission directions of the optical detection signal and the optical reflection signal. The lens unit 50 can include one or plural optical components, which may be any types of concave lenses and/or convex lenses for light divergence and light convergence; variation of the optical components of the lens unit 50 can depend on the design demand, and a detailed description is omitted herein for simplicity. The first sheltering unit 52 can be used to seal the first opening 36, so as to prevent the lens unit 50 from be polluted by dust for preferred identification accuracy of the optical identification assembly 42.

The optical emitter 46 and the optical receiver 48 can be preferably near the lens unit 50, to ensure a planar gap between the optical emitter 46 and the optical receiver 48 can be equal to zero to avoid unintended refraction of the optical signals. Therefore, the wearable device 10 can further include a resiliently supporting component 56 disposed under the hard circuit boards or the soft circuit boards, and used to provide an upwardly supporting force to abut the optical emitter 46 and the optical receiver 48 against the lens unit 50. Moreover, the detection module 16 can further include a force feedback unit 58 connected to the optical identification assembly 42 and used to move the optical identification assembly 42. The force feedback unit 58 may utilize material property of piezoelectric material, elastic material, or magnetic material to provide a position recovering function. If the optical identification assembly 42 is pressed, the force feedback unit 58 is compressed and deformed, and the optical identification assembly 42 may be moved downwardly in a slight manner; if an external force applied for the optical identification assembly 42 is decreased or removed, the position recovering function of the force feedback unit 58 can upwardly push the optical identification assembly 42 so that the user can feel a reaction force to enrich the operation experience.

If a part of the outer casing 22 relevant to the information transmission unit 40 is made of non-metallic material, such as plastic material, a wireless signal of the information transmission unit 40 can directly pierce through the outer casing 22. If the outer casing 22 is made by metallic material due to a specific design demand, the outer casing 22 can include a second opening 60, and a position of the second opening 60 can correspond to a position of the information transmission unit 40. In the meantime, the outer casing 22 can further include a second sheltering unit 62 used to seal the second opening 60; the second sheltering unit 62 can be made of non-metallic material, and the wireless signal of the information transmission unit 40 can pierce through the second sheltering unit 62 so as to establish wireless connection with the external device.

The main body 26 of the inner casing 20 can include a hole 64, and the wearable device 10 can further include a charging assembly 66. The charging assembly 66 can include a pin 68, a first magnetic component 70 and other components. The pin 68 can be inserted into the hole 64 and electrically connected with a conductive unit 72 of the external charger 12. The charging assembly 66 can utilize the first magnetic component 70 to contact or be close to a second magnetic component 74 of the external charger 12 for generating a magnetic attraction force, so as to constrain a relative position between the wearable device 10 and the external charger 12.

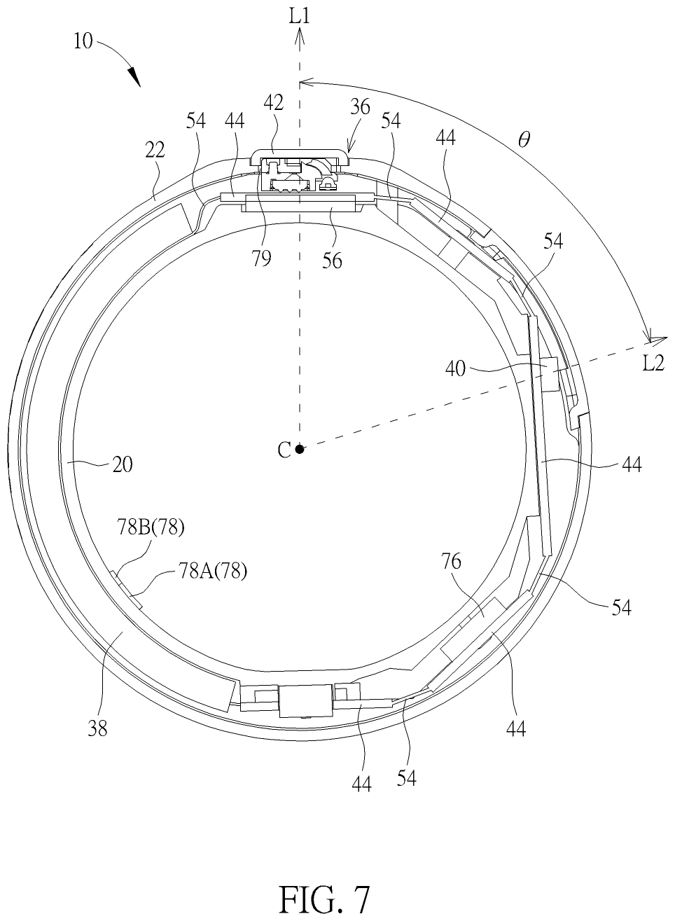

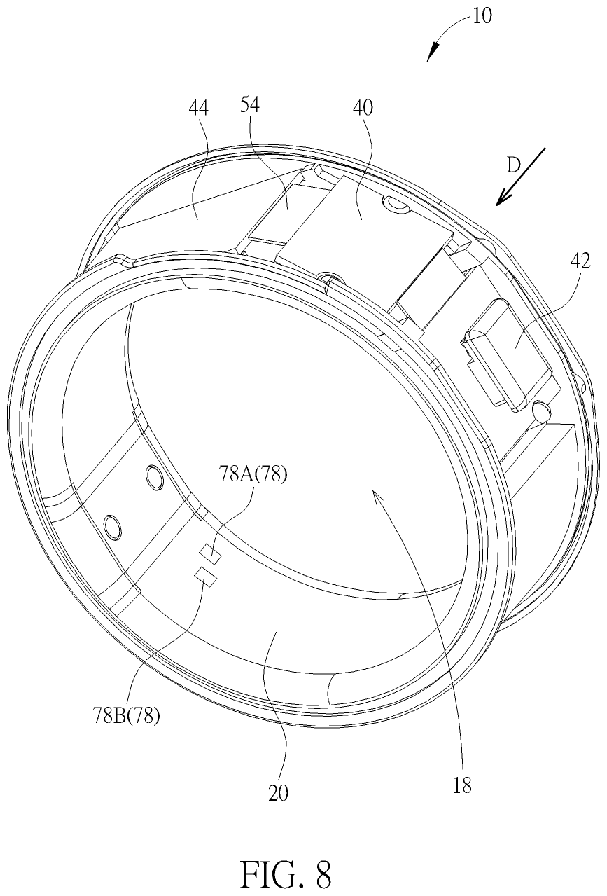

Please refer to FIG. 7 and FIG. 8. FIG. 7 is a section view of the wearable device 10 according to the embodiment of the present invention. FIG. 8 is a diagram of a part of the wearable device 10 according to the embodiment of the present invention. The wearable device 10 may further include a gravity sensor 76 and a touch sensor 78, respectively disposed on relevant regions of the circuit board 44. The gravity sensor 76 can be disposed on the main body 26 of the inner casing 20 and be covered by the outer casing 22, and used to detect acceleration variation of the wearable device 10; if the user falls down, the gravity sensor 76 can detect the acceleration variation and output a warning reminder. The touch sensor 78 can be disposed on an inner surface of the inner casing 20 facing the accommodating space 18. The touch sensor 78 can be a capacitive detector, and a number of the touch sensor 78 can depend on the design demand. Each of the touch sensors 78 can detect whether the organism is accommodated inside the case 14. A plurality of touch sensors 78 can be arranged along a horizontal direction of the case 14, such as the touch sensors 78A and 78B shown in FIG. 7, and used to detect a moving direction of the case 14 relative to the organism.

For example, if the touch sensor 78A detects the organism earlier than the touch sensor 78B, the wearable device 10 can determine that the organism is moved into the accommodating space 18 of the case 14 in an arrow direction D; if the touch sensor 78A detects the organism later than the touch sensor 78B, the wearable device 10 can determine that the organism is moved into the accommodating space 18 of the case 14 in another direction opposite to the arrow direction D. Therefore, the present invention can detect whether the wearable device 10 is worn by the user, and detect a wearing direction of the wearable device 10. In addition, optical identification assembly 42 can be optionally integrated with the photoplethysmography (PPG) sensor and the pulse oximetry (SpO2) sensor, so as to provide the heart rate detection function and the blood oxygen detection function. The wearable device 10 can be further integrated with the gravity sensor 76 and the touch sensor 78, and accordingly provide the motion detection function of detecting the sudden motion and the hand detection function of detecting the wearable device 10 is worn on the right hand or the left hand of the user.

It should be mentioned that an included angle θ between a first virtual extension line L1 from the optical identification assembly 42 to a center C of the case 14 and a second virtual extension line L2 from the information transmission unit 40 to the center C can be ranged between sixty-five degrees and eighty degrees; this structural design can ensure that the wireless transmission function of the information transmission unit 40 is not affected when the user operates the optical identification assembly 42, so that the wearable device 10 can provide the optimal operation experience. An actual value of the included angle θ may be varied in accordance with information receiving and emitting function of the information transmission unit 40 and/or configuration requirements of the wearable device 10, and the detailed description is omitted herein for simplicity.

The first sheltering unit 52 of the optical identification assembly 42 may be partly protruded from the first opening 36 for convenient pressing, and therefore the detection module 16 can optionally include a waterproof unit 79 filled inside a gap between the optical identification assembly 42 and the first opening 36 for avoid liquid leakage.

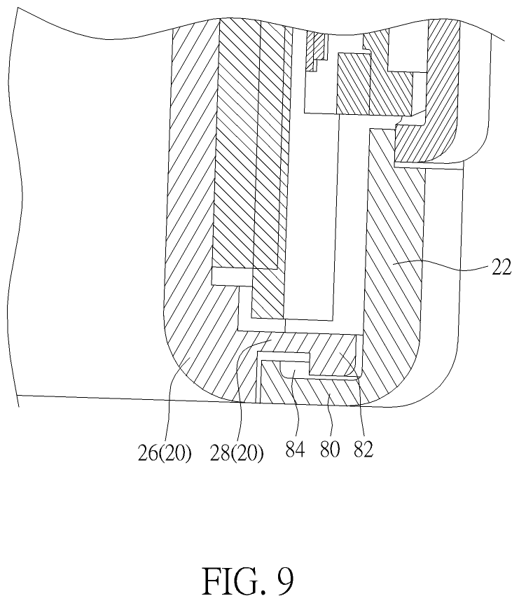

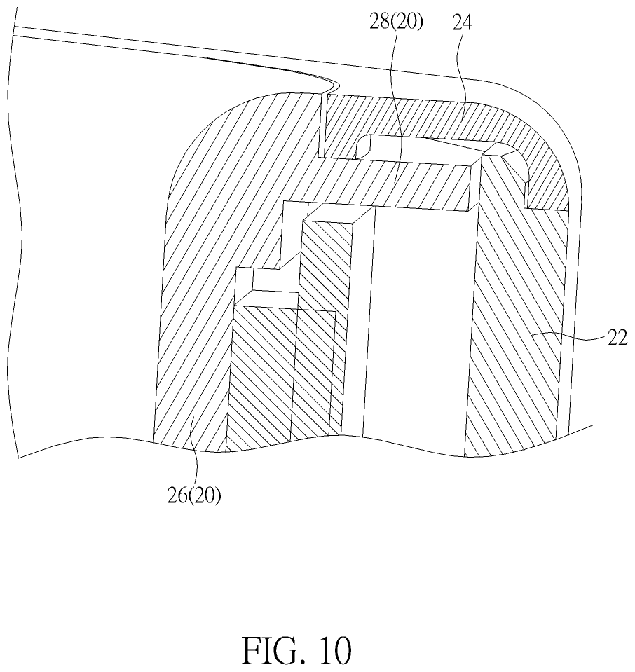

Please refer to FIG. 2, FIG. 9 and FIG. 10. FIG. 9 is a section view of the wearable device 10 on a section line A-A shown in FIG. 2. FIG. 10 is a section view of the wearable device 10 on a section line B-B shown in FIG. 2. As shown in FIG. 9, a first engaging portion 80 can be disposed on a lateral side of the outer casing 22, and a second engaging component 82 can be disposed on the lateral wall 28 of the inner casing 20. The first engaging portion 80 can be engaged with an outer edge of the second engaging component 82 to prevent the outer casing 22 and the inner casing 20 from separation. A gap may be formed between the first engaging portion 80 and the second engaging component 82, and can be filled by waterproof material 84 to avoid the liquid leakage. As shown in FIG. 10, the lateral cover 24 can be disposed on another lateral side of the outer casing 22 that does not have the first engaging portion 80, and can be further engaged with related structures of the inner casing 20 and the outer casing 22; the gap between the inner casing 20 and the lateral cover 24 can be filled by the waterproof material 84 to avoid the liquid leakage.

In conclusion, the wearable device of the present invention can be worn on the finger, the wrist or the forehead of the user for executing the optical identification function, and can optionally include the information transmission unit, the energy storage unit, the gravity sensor and the touch sensor to provide various detection functions. The optical identification assembly can detect rotation of the wearable device, and further be integrated with the photoplethysmography sensor and the pulse oximetry sensor for the biological detection.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A wearable device with an optical identification function, the wearable device comprising:
a case having an accommodating space where inside an organism is accommodated, the case comprising:
an inner casing comprising a main body and a lateral wall, the main body being an annular structure to form the accommodating space, the lateral wall being disposed on a lateral side of the main body, the main body having a first installation area, a second installation area and a third installation area; and
an outer casing disposed around the inner casing and abutting against the lateral wall, the outer casing having a first opening; and
a detection module disposed inside the case, the detection module comprising:
an energy storage unit located on the first installation area;
an information transmission unit located on the second installation area and adapted to transmit identification information; and
an optical identification assembly located on the third installation area and electrically connected with the energy storage unit and the information transmission unit, the optical identification assembly aligning with the first opening and being adapted to output an optical detection signal toward the organism through the first opening and further to receive and transform an optical reflection signal from the organism into the identification information.

2. The wearable device of claim 1, wherein the case further comprises a lateral cover adapted to cover a space formed between the inner casing and the outer casing assembled with each other.

3. The wearable device of claim 1, wherein the optical identification assembly comprises an optical emitter, an optical receiver, a lens unit and a first sheltering unit, the lens unit is disposed above the optical emitter and the optical receiver and used to constrain a transmission direction of the optical detection signal and the optical reflection signal, the first opening is sealed by the first sheltering unit.

4. The wearable device of claim 1, wherein the wearable device further comprises a circuit board disposed on the inner casing and covered by the outer casing, the circuit board has several regions respectively corresponding to the first installation area, the second installation area and the third installation area.

5. The wearable device of claim 4, wherein the detection module is disposed on a surface of the circuit board to face toward one of inside and outside of the case, or is disposed on two opposite surfaces of the circuit board to face toward the inside and the outside of the case.

6. The wearable device of claim 4, wherein the circuit board is a plurality of hard circuit boards electrically connected with each other via transmission cables, or a plurality of soft circuit boards electrically connected with each other via the transmission cables, or the plurality of hard circuit boards and the plurality of soft circuit boards electrically connected with each other via the transmission cables.

7. The wearable device of claim 6, wherein the wearable device further comprises a resiliently supporting component disposed under the soft circuit boards.

8. The wearable device of claim 1, wherein the outer casing is made of metallic material, and the outer casing further comprises a second opening corresponding to the information transmission unit.

9. The wearable device of claim 8, wherein the outer casing further comprises a second sheltering unit adapted to seal the second opening.

10. The wearable device of claim 1, wherein a part of the outer casing that corresponds to the information transmission unit is made of non-metallic material.

11. The wearable device of claim 1, wherein the detection module further comprises a waterproof unit filled inside a gap between the optical identification assembly and the first opening.

12. The wearable device of claim 11, wherein the detection module further comprises a force feedback unit electrically connected with the optical identification assembly and adapted to move the optical identification assembly.

13. The wearable device of claim 12, wherein the optical identification assembly is moved by material property of piezoelectric material, elastic material, or magnetic material of the force feedback unit.

14. The wearable device of claim 1, wherein the main body of the inner casing comprises a hole, the wearable device further comprises a charging assembly, at least one pin of the charging assembly is inserted into the hole, a first magnetic component of the charging assembly is matched with a second magnetic component of an external charger to constrain a relative position between the wearable device and the external charger.

15. The wearable device of claim 1, wherein the wearable device further comprises a gravity sensor disposed one the main body and adapted to detect acceleration variation of the wearable device.

16. The wearable device of claim 1, wherein the wearable device further comprises a touch sensor disposed inside the inner casing and adapted to detect whether the organism is accommodated inside the case.

17. The wearable device of claim 1, wherein the wearable device further comprises a plurality of touch sensors arranged inside the inner casing along a horizontal direction of the case and adapted to detect a moving direction of the case relative to the organism.

18. The wearable device of claim 1, wherein an included angle between a first virtual extension line from the optical identification assembly to a center of the case and a second virtual extension line from the information transmission unit to the center is ranged between sixty-five degrees and eighty degrees.

19. The wearable device of claim 1, wherein a first engaging portion is disposed on a lateral side of the outer casing, a second engaging component is disposed on the lateral wall of the inner casing, and the first engaging portion is engaged with an outer edge of the second engaging component to avoid separation.

20. The wearable device of claim 19, wherein waterproof material is filled between the first engaging portion and the second engaging component.
